(19) United States
(12) Patent Application Publication
(10) Pub. No.: US 2023/0197285 A1
(43) Pub. Date: Jun. 22, 2023

(54) PATIENT CONDITION PREDICTION APPARATUS, PATIENT CONDITION PREDICTION METHOD, AND COMPUTER PROGRAM

(71) Applicant: NEC Corporation, Minato-ku, Tokyo (JP)

(72) Inventors: Masahiro HAYASHITANI, Tokyo (JP); Eiji YUMOTO, Tokyo (JP); Toshinori HOSOI, Tokyo (JP); Masahiro KUBO, Tokyo (JP)

(73) Assignee: NEC Corporation, Minato-ku, Tokyo (JP)

(21) Appl. No.: 17/768,970

(22) PCT Filed: Oct. 31, 2019
(86) PCT No.: PCT/JP2019/042830

§ 371 Date: Apr. 14, 2022

(51) Int. Cl. G16H 50/50; G16H 10/60; G16H 20/00

(52) U.S. Cl. CPC G16H 50/50; G16H 10/60; G16H 20/00

(57) ABSTRACT

A patient condition prediction apparatus includes: an acquisition unit that obtains patient data, which are information about a patient; a selection unit that selects one predictive model from a plurality of predictive models for predicting a change in a patient condition that is a condition of the patient, on the basis of the patient data; and a prediction unit that predicts a change in the patient condition in the future by using the one predictive model. This makes it possible to predict a change in the patient condition by using an appropriate predictive model.

### TECHNICAL FIELD

The present invention relates to a patient condition prediction apparatus, a patient condition prediction method, and a computer program that predict a patient's condition.

### BACKGROUND ART

A known apparatus of this type predicts the condition of a patient (e.g., a patient in a hospital, etc.). For example, Patent Literature 1 discloses a technique/technology of generating a predictive model for predicting an occurrence of a predetermined event from patients' conditions classified into a plurality of clusters.

It is also known, as a technique/technology that uses the predictive model, that a plurality of predictive models are used. For example, Patent Literature 2 discloses a technique/technology of preferentially selecting a predictive model with higher evaluation from a plurality of predictive models.

As another related technique/technology, Patent Literature 3 discloses a technique/technology of predicting that a patient will develop a disease within a reference period and notifying the patient of that. Patent Literature 4 discloses a technique/technology of deriving information about a disease from a selected model.

### CITATION LIST

### Patent Literature

Patent Literature 1: JP2018-180993A
Patent Literature 2: International Publication WO2016/148107 pamphlet
Patent Literature 3: JP2019-016235A
Patent Literature 4: JP2018-200567A

### SUMMARY

### Technical Problem

When predicting the condition of a patient with a predictive model, it is preferable to use a predictive model that is suitable for that patient. In other words, the use of a predictive model that is not suitable for the patient may result in an inaccurate prediction of the patient's condition.

However, it is difficult to prepare a predictive model that is suitable for all the patients beforehand, because each of the patients has different characteristics. Furthermore, even if a plurality of types of predictive models are prepared, it is not easy to select a predictive model that is suitable for a patient from them. That is, each of the above-described Patent Literatures has room for improvement in terms of accurately predicting a patient's condition.

In view of the problems described above, it is therefore an example object of the present invention to provide a patient condition prediction apparatus, a patient condition prediction method, and a computer program that are configured to predict a change in a patient's condition by using an appropriate predictive model.

### Solution to Problem

A patient condition prediction apparatus according to an example aspect of the present invention includes: an acquisition unit that obtains patient data, which are information about a patient; a selection unit that selects one predictive model from a plurality of predictive models for predicting a change in a patient condition that is a condition of the patient, on the basis of the patient data; and a prediction unit that predicts a change in the patient condition in the future by using the one predictive model.

A patient condition prediction method according to an example aspect of the present invention includes: obtaining patient data, which are information about a patient; selecting one predictive model from a plurality of predictive models for predicting a change in a patient condition that is a condition of the patient, on the basis of the patient data; and predicting a change in the patient condition in the future by using the one predictive model.

A computer program according to an example aspect of the present invention operates a computer: to obtain patient data, which are information about a patient; to select one predictive model from a plurality of predictive models for predicting a change in a patient condition that is a condition of the patient, on the basis of the patient data; and to predict a change in the patient condition in the future by using the one predictive model.

### Effect of the Invention

According to the patient condition prediction apparatus, the patient condition prediction method, and the computer program according to the respective example embodiments described above, it is possible to accurately predict a change in a patient's condition by using an appropriate predictive model.

### BRIEF DESCRIPTION OF DRAWINGS

FIG. 1 is a block diagram illustrating an overall configuration of a patient condition prediction apparatus according to a first example embodiment.

FIG. 2 is a block diagram illustrating a hardware configuration of the patient condition prediction apparatus according to the first example embodiment.

FIG. 3 is a flowchart illustrating a flow of operation of the patient condition prediction apparatus according to the first example embodiment.

FIG. 4 is a diagram illustrating an example of a method of selecting a predictive model on the basis patient data.

FIG. 5 is a chart illustrating an example of a method of predicting a patient condition by using the predictive model.

FIG. 6 is a flowchart illustrating a flow of operation of a patient condition prediction apparatus according to a second example embodiment.

FIG. 7 is a chart illustrating an example of a method of determining a risk of complications from the predicted patient condition.

### DESCRIPTION OF EXAMPLE EMBODIMENTS

With reference to the drawings, a patient condition prediction apparatus, a patient condition prediction method, and a computer program according to example embodiments will be described below.

### <First Example Embodiment>

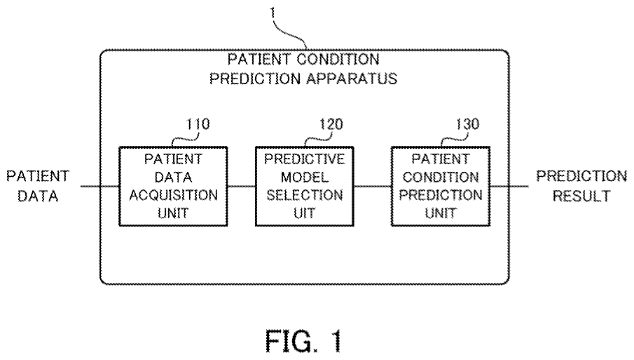

A patient condition prediction apparatus according to a first example embodiment will be described with reference to FIG. 1 to FIG. 5.

### (Apparatus Configuration)

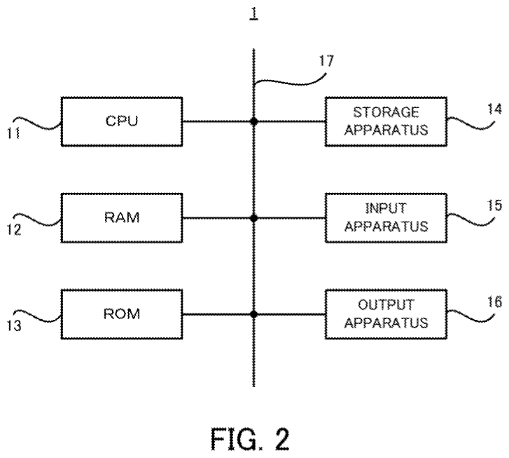

Firstly, a configuration of the patient condition prediction apparatus according to the first example embodiment will be described with reference to FIG. 1 and FIG. 2. FIG. 1 is a block diagram illustrating an overall configuration of the patient condition prediction apparatus according to the first example embodiment. FIG. 2 is a block diagram illustrating a hardware configuration of the patient condition prediction apparatus according to the first example embodiment.

In FIG. 1, a patient condition prediction apparatus 1 according to the first example embodiment is an apparatus that predicts a change in a patient's condition (i.e., a “patient condition”). Incidentally, the “patient condition” here is a term that represents a state of a patient's symptom. For example, it quantitatively indicates a state of recovery after a surgery of an inpatient or the like (more specifically, whether a symptom is improved, or whether the inpatient can perform activities of daily living, etc.). The patient condition prediction apparatus 1 includes, as main components, a patient data acquisition unit 110, a predictive model selection unit 120, and a patient condition prediction unit 130.

The patient data acquisition unit 110 is configured to obtain patient data, which are information about a patient. The “patient data” are data that can influence a change in the patient condition in the future, such as a patient's attribute, various data about a patient measured in a hospital, and an index calculated from the patient condition. Specific examples of the patient data include: general vital signs (blood pressure, pulse, body temperature, etc.); various indexes calculated from the patient's condition such as FIM (Functional Independence Measure), BI (Barthel Index), NIHSS (National Institute of Health Stroke Scale), MMT (Manual Muscle Test), JCS (Japan Coma Scale), and SpO2 (percutaneous arterial blood oxygen saturation), as well as information about a patient's hospitalization period. Incidentally, a detailed description of a specific method of obtaining (or method of calculating) the patient data will be omitted here because it is possible to appropriately adopt the existing techniques. The patient data obtained by the patient data acquisition unit 110 is configured to be outputted to the predictive model selection unit 120.

The predictive model selection unit 120 is configured to select a predictive model for predicting the patient condition on the basis of the patient data obtained by the patient data acquisition unit 110. More specifically, the predictive model selection unit 120 stores a plurality of types of predictive models in advance, and selects one predictive model that is suitable for the patient data (in other words, a predictive model that allows more accurate prediction of the patient condition of the patient) from the plurality of types of predictive models. A specific method of selecting the predictive model will be discussed in detail later. The “predictive model” is an arithmetic model used to predict a future patient condition, and is generated, for example, by machine learning or the like. The technique of machine learning is not particularly limited, and a suitable technique may be used in accordance with the patient data to be used or the like. Furthermore, each of the plurality of predictive models may be generated in the same manner or in different manners. A result of the selection by the predictive model selection unit 120 is configured to be outputted to the patient condition prediction unit 130.

The patient condition prediction unit 130 is configured to predict a future patient condition by using the predictive model selected by the predictive model selecting unit 120. Specifically, the patient condition prediction unit 130 inputs the patient data (which may include past or current patient conditions) into the predictive model, and obtains the future patient condition as its output. A more specific method of predicting the patient condition will be described in detail later. The patient condition predicted by the patient condition prediction unit 130 is configured to be outputted to an external apparatus (e.g., a display, etc.).

As illustrated in FIG. 2, the patient condition prediction apparatus 1 according to this example embodiment includes a CPU (Central Processing Unit) 11, a RAM (Random Access Memory) 12, a ROM (Read Only Memory) 13, and a storage apparatus 14. The patient condition prediction apparatus 1 may also include an input apparatus 15 and an output apparatus 16. The CPU 11, the RAM 12, the ROM 13, the storage apparatus 14, the input apparatus 15, and the output apparatus 16 are connected through a data bus 17.

The CPU 11 reads a computer program. For example, the CPU 11 may read a computer program stored by at least one of the RAM 12, the ROM 13 and the storage apparatus 14. For example, the CPU 11 may read a computer program stored by a computer readable recording medium, by using a not-illustrated recording medium read apparatus. The CPU 11 may obtain (i.e., read) a computer program from a not-illustrated apparatus located outside the patient condition prediction apparatus 1, through a network interface. The CPU 11 controls the RAM 12, the storage apparatus 14, the input apparatus 15, and the output apparatus 16 by executing the read computer program. Especially in this example embodiment, when the CPU 11 executes the read computer program, a functional block for predicting the patient condition is implemented in the CPU 11. The patient data acquisition unit 110, the predictive model selection unit 120, and the patient condition prediction unit 130 described above are implemented, for example, in this CPU 11.

The RAM 12 temporarily stores the computer program to be executed by the CPU 11. The RAM 12 temporarily stores the data that is temporarily used by the CPU 11 when the CPU 11 executes the computer program. The RAM 12 may be, for example, D-RAM (Dynamic RAM).

The ROM 13 stores the computer program to be executed by the CPU 11. The ROM 13 may otherwise store fixed data. The ROM 13 may be, for example, a P-ROM (Programmable ROM).

The storage apparatus 14 stores the data that is stored for a long term by the patient condition prediction apparatus 1. The storage apparatus 14 may operate as a temporary storage apparatus of the CPU 11. The storage apparatus 14 may include, for example, at least one of a hard disk apparatus, a magneto-optical disk apparatus, an SSD (Solid State Drive), and a disk array apparatus.

The input apparatus 15 is an apparatus that receives an input instruction from a user of the patient condition prediction apparatus 1. The input apparatus 15 may include, for example, at least one of a keyboard, a mouse, a touch panel, a smart phone, and a tablet.

The output apparatus 16 is an apparatus that outputs information about the patient condition prediction apparatus 1 to the outside. For example, the output apparatus 16 may be a display apparatus that is configured to display the information about the patient condition prediction apparatus 1.

### (Description of Operation)

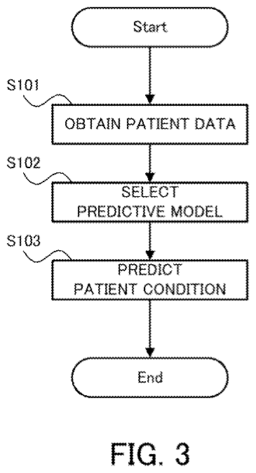

Next, with reference to FIG. 3, a flow of operation of the patient condition prediction apparatus 1 according to the first example embodiment will be described. FIG. 3 is a flow chart illustrating the flow of operation of the patient condition prediction apparatus according to the first example embodiment.

As illustrated in FIG. 3, in operation of the patient condition prediction apparatus 1 according to the first example embodiment, the patient data acquisition unit 110 firstly obtains the patient data (step S101). Incidentally, the patient data obtained here may be not only current ones but also those obtained in the past (in other words, past patient data that are accumulated).

Subsequently, the predictive model selection unit 120 selects the predictive model on the basis of the patient data obtained by the patient data acquisition unit 110 (step S102). When a plurality of types of patient data are obtained, one type of them may be used to obtain the predictive model, or a plurality of types may be used (or combined) to obtain the predictive model.

Subsequently, the patient condition prediction unit 130 predicts the patient condition by using the predictive model selected by the predictive model selecting unit 120 (step S103). The patient condition predicted here indicates the future patient condition, and may allow determination of a state of the patient's symptom in a few days or a risk of complications.

### (Selection of Predictive Model)

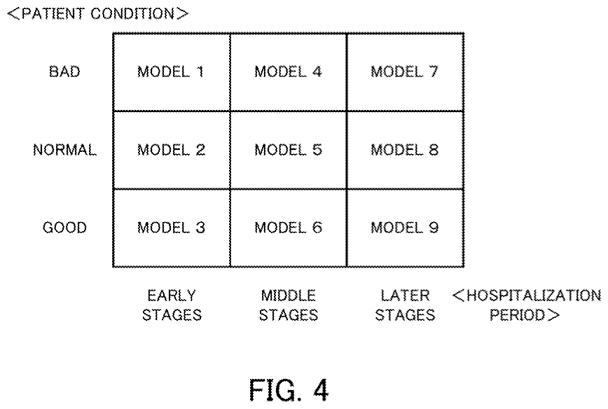

Next, with reference to FIG. 4, a specific method of selecting the predictive model (i.e., details of the step S102 in FIG. 3) will be described. FIG. 4 is a diagram illustrating an example of the method of selecting the predictive model on the basis of the patient data.

As illustrated in FIG. 4, the predictive model selection unit 120 may select the predictive model on the basis of the “(past or current) patient condition” and the “hospitalization period” obtained as the patient data. For example, the predictive model selection unit 120 may select the predictive model on the basis of both a degree of the patient condition and a timing in the hospitalization period (i.e., how long a period has elapsed when viewed from an entire hospitalization period). Here, any of Models 1 to 9 is selected depending on whether the patient condition is “good,” “normal,” or “bad,” and whether the hospitalization period is “in early stages,” “in middle stages,” or “in later stages.” For example, when the patient's hospitalization period is “in early stages” and the patient condition is “bad”, Model 1 is selected as the predictive model that is suitable for that patient. When the patient's hospitalization period is “in middle stages” and the patient condition is “normal”, Model 5 is selected as the predictive model that is suitable for that patient. When the patient's hospitalization period is “in later stages” and the patient condition is “good”, Model 9 is selected as the predictive model that is suitable for that patient.

As described above, the predictive model is selected on the basis of the patient data obtained by the patient data acquisition unit 110. Incidentally, the selection method illustrated in FIG. 4 is merely an example, and the predictive model may be selected by using other techniques.

For example, the predictive model may be selected on the basis of only one of the patient condition and the hospitalization period, or the predictive model may be selected on the basis of one or more other factors in addition to the patient condition and the hospitalization period.

### (Prediction of Patient Condition)

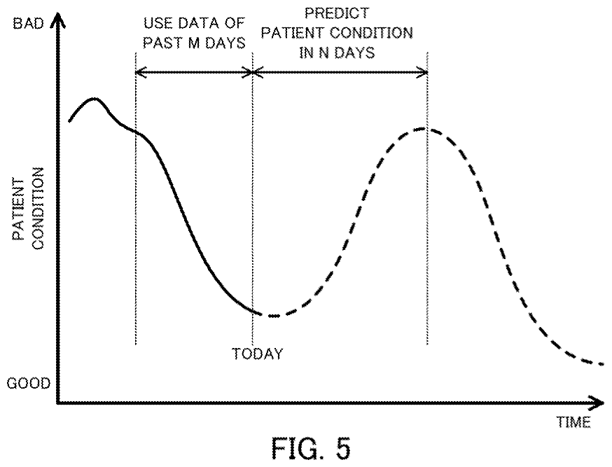

Next, with reference to FIG. 5, a method of predicting the patient conditions by using the predictive model (i.e., details of the step S103 in FIG. 3) will be described. FIG. 5 is a chart illustrating an example of the method of predicting the patient condition by using the predictive model.

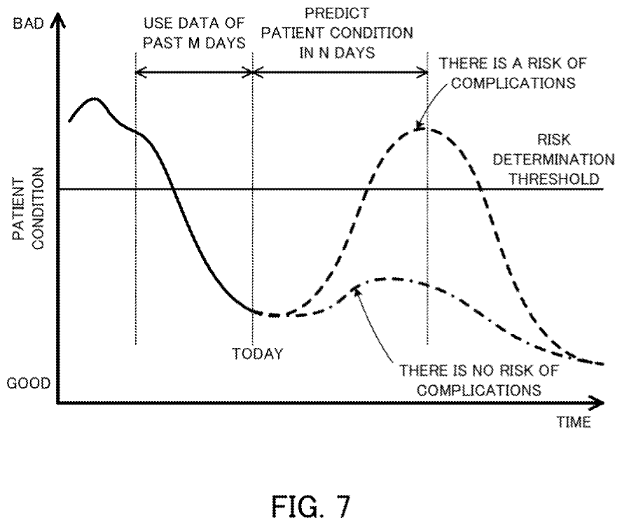

As illustrated in FIG. 7, the predictive model may allow prediction of the patient condition in N days by using the patient condition of the past M days (where “M” and “N” are natural numbers). In this case, the patient condition prediction unit 130 inputs data indicating the patient condition of the past M days into the predictive model selected by the predictive model selecting unit 120. Then, data indicating the patient condition in N days from today are outputted from the predictive model as a prediction result. By such an operation, the patient condition predictor 130 predicts the future patient condition.

In this example embodiment, a plurality of predictive models in which the values of “M” and “N” described above are appropriately defined are prepared in advance, and the predictive model selection unit 120 may select one predictive model from them on the basis of the patient data. In other words, the predictive model selection unit 120 may determine appropriate “M” and “N” on the basis of the patient data. In this way, the selection of the predictive model makes it possible to more appropriately predict the future patient condition. For example, if the patient condition tends to be stable when the hospitalization period is relatively long, accurate prediction can be performed by increasing M (i.e., by selecting a predictive model with a larger M) as the hospitalization period is longer. In addition, if the patient condition tends to be stable when the patient is young in age, accurate prediction can be performed by increasing M (i.e., by selecting a predictive model with a larger M) as the patient is younger in age. In addition, in a situation where the patient condition needs to be grasped for a longer period, appropriate prediction can be performed by increasing N (i.e., by selecting a predictive model with a larger N).

### (Technical Effect)

Next, a technical effect obtained by the patient condition prediction apparatus 1 according to the first example embodiment will be described.

As described in FIG. 1 to FIG. 5, according to the patient condition prediction apparatus 1 in the first example embodiment, the patient condition is predicted by using the predictive model selected on the basis of the patient data. For this reason, it is possible to predict the patient condition more accurately than when the patient condition is predicted by using only one predictive model at each time. That is to say, the prediction result is more accurate because the patient condition can be predicted by using the predictive model that is suitable for each patient.

### <Second Example Embodiment>

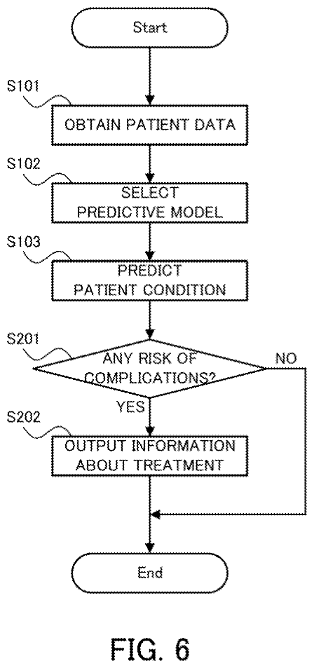

Next, a patient condition prediction apparatus according to a second example embodiment will be described with reference to FIG. 6 and FIG. 7. The second example embodiment is partially different from the first example embodiment described above only in the configuration and operation, and is substantially the same in the other parts. Therefore, the parts that differ from the first example embodiment described above will be described below, and the other overlapping parts will not be described.

### (Description of Operation)

Firstly, with reference to FIG. 6, a flow of operation of the patient condition prediction apparatus 1 according to the second example embodiment will be described. FIG. 6 is a flow chart illustrating the flow of operation of the patient condition prediction apparatus according to the second example embodiment. Incidentally, in FIG. 6, the same steps as those illustrated in FIG. 3 carry the same reference numerals.

As illustrated in FIG. 6, in operation of the patient condition prediction apparatus 1 according to the second example embodiment, as in the first example embodiment, the patient data acquisition unit 110 obtains the patient data (step S101), the predictive model selection unit 120 selects the predictive model on the basis of the obtained patient data (step S102), and the patient condition prediction unit 130 predicts the patient condition by using the selected predictive model (step S103).

Then, in the second example embodiment, the patient condition prediction unit 130 determines whether or not there is a risk at which complications occur in the patient (hereinafter referred to as a “risk of complications”) on the basis of the predicted patient condition (step S201). A specific method of determining the risk of complications will be described in detail later.

When it is determined that there is a risk of complications (the step S201: YES), the patient condition predictor 130 outputs information about treatment (care) for the patient (typically, information about treatment to reduce the risk of complications) (step S202). More specifically, the patient condition prediction unit 130 predicts the complications that may occur in the patient, specifies treatment effective to suppress the occurrence of the complications, and notifies a medical staff or the like of the content of the specified treatment. Incidentally, the information to be outputted may be changed depending on the degree of the risk of complications. For example, when the risk of complications is relatively low, the number of types of treatment to be outputted may be reduced and only easy-to-practice treatment (e.g., oral care, bed angle up, etc.) may be outputted. On the other hand, when the risk of complications is relatively high, the number of types of treatment to be outputted may be increased and difficult-to-practice treatment (e.g., breathing exercise, abdominal pressure breathing training, etc.) may be outputted.

When it is determined that there is no risk of complications (the step S201: NO), the step S202 described above is omitted. That is, the information about treatment to reduce the risk of complications is not outputted.

### (Determination of Risk of Complications)

Next, with reference to FIG. 7, a method of determining the risk of complications (i.e., details of the step S201 in FIG. 6) will be described. FIG. 7 is a chart illustrating an example of the method of determining the risk of complications from the predicted patient condition.

As illustrated in FIG. 7, the risk of complications may be determined on the basis of a state of the patient condition predicted by the patient condition predictor 130. Specifically, when the patient condition exceeds a risk determination threshold and deflects to a worse side, the patient condition prediction unit 130 may determine that there is a risk of complications (see a broken line in the figure). On the other hand, when the patient condition does not exceed the risk determination threshold, the patient condition predictor 130 may determine that there is no risk of complications (see an alternate long and short dashed line in the figure). Incidentally, the “risk determination threshold” here is a threshold set in advance to determine whether or not there is a risk of complications, and is a value calculated, for example, on the basis of data about the past complications or the like. Setting a plurality of risk determination thresholds may allow step-by-step prediction of the risk of complications (i.e., prediction of the degree of the risk of complications). Incidentally, the method of using the risk determination threshold is merely an example, and it may be determined whether or not there is a risk of complications by a determination method other than the threshold.

### (Technical Effect)

Next, a technical effect obtained by the patient condition prediction apparatus 1 according to the second example embodiment will be described.

As described in FIG. 6 and FIG. 7, according to the patient condition prediction apparatus 1 in the second example embodiment, the risk of complications is predicted on the basis of the predicted patient information. Therefore, it is possible to predict the occurrence of complications (in other words, the deterioration of the patient's condition) in advance. Furthermore, when the occurrence of complications is predicted, the information about treatment to reduce the risk of complications is outputted. Therefore, it is possible to efficiently prevent the occurrence of complications.

The occurrence of complications is a major cause of delayed discharge in healthcare facilities. Therefore, it is possible to avoid the occurrence of delayed discharge by preventing the occurrence of complications. As a result, beneficial effects can be obtained even for problems such as insufficient number of sickbeds.

The treatment to reduce the risk of complications may be outputted for all the patients, but in that case, a medical staff is required to respond to all the patients, and this may significantly increase their workload. In this example embodiment, however, the information about treatment is outputted depending on the presence or absence of the risk of complications, and thus, the medical staff can efficiently treat the patient to be treated. Therefore, the workload of the medical staff can be reduced.

### <Supplementary Notes>

With respect to the example embodiment described above, the following Supplementary Notes will be further disclosed.

### (Supplementary Note 1)

A patient condition prediction apparatus described in Supplementary Note 1 is a patient condition prediction apparatus including: an acquisition unit that obtains patient data, which are information about a patient; a selection unit that selects one predictive model from a plurality of predictive models for predicting a change in a patient condition that is a condition of the patient, on the basis of the patient data; and a prediction unit that predicts a change in the patient condition in the future by using the one predictive model.

### (Supplementary Note 2)

A patient condition prediction apparatus described in Supplementary Note 2 is the patient condition prediction apparatus described in Supplementary Note 1, wherein the prediction unit predicts a risk of complications indicating a possibility of the patient developing complications, on the basis of the predicted change in the patient condition in the future.

(Supplementary Note 3) A patient condition prediction apparatus described in Supplementary Note 3 is the patient condition prediction apparatus described in Supplementary Note 2, wherein the prediction unit outputs information about treatment for the patient on the basis of the predicted risk of complications.

### (Supplementary Note 4)

A patient condition prediction apparatus described in Supplementary Note 4 is the patient condition prediction apparatus described in any one of Supplementary Notes 1 to 3, wherein each of the plurality of predictive models is a model that allows prediction of the change in the patient condition in the future by using the patient condition in a first period that is defined for each of the predictive models and that is in the past.

### (Supplementary Note 5)

A patient condition prediction apparatus described in Supplementary Note 5 is the patient condition prediction apparatus described in Supplementary Note 4, wherein the selection unit selects a model in which the first period is longer as a hospitalization period of the patient is longer.

### (Supplementary Note 6)

A patient condition prediction apparatus described in Supplementary Note 6 is the patient condition prediction apparatus described in Supplementary Note 4 or 5, wherein the selection unit selects a model in which the first period is longer as the patient is younger in age.

### (Supplementary Note 7)

A patient condition prediction apparatus described in Supplementary Note 7 is the patient condition prediction apparatus described in any one of Supplementary Notes 1 to 6, wherein each of the plurality of predictive models is a model that allows prediction of the change in the patient condition in a second period that is defined for each of the predictive models and that is in the future.

### (Supplementary Note 8)

A patient condition prediction apparatus described in Supplementary Note 8 is the patient condition prediction apparatus described in Supplementary Note 7, wherein the selection unit selects a model in which the second period is longer as the patient condition needs to be grasped for a longer period.

### (Supplementary Note 9)

A patient condition prediction apparatus described in Supplementary Note 9 is the patient condition prediction apparatus described in any one of Supplementary Notes 1 to 8, wherein the patient data includes an index that is defined by a degree of activities that can be performed by the patient.

### (Supplementary Note 10)

A patient condition prediction apparatus described in Supplementary Note 10 is the patient condition prediction apparatus described in any one of Supplementary Notes 1 to 9, wherein the patient data includes information about a hospitalization period of the patient.

### (Supplementary Note 11)

A patient condition prediction apparatus described in Supplementary Note 11 is the patient condition prediction apparatus described in any one of Supplementary Notes 1 to 10, wherein the patient data includes information about vital signs of the patient.

### (Supplementary Note 12)

A patient condition prediction method according to Supplementary Note 12 is a patient condition prediction method including: obtaining patient data, which are information about a patient; selecting one predictive model from a plurality of predictive models for predicting a change in a patient condition that is a condition of the patient, on the basis of the patient data; and predicting a change in the patient condition in the future by using the one predictive model.

### (Supplementary Note 13)

A computer program according to Supplementary Note 13 is a computer program that operates a computer: to obtain patient data, which are information about a patient; to select one predictive model from a plurality of predictive models for predicting a change in a patient condition that is a condition of the patient, on the basis of the patient data; and to predict a change in the patient condition in the future by using the one predictive model.

### (Supplementary Note 14)

A recording medium described in Supplementary Note 14 is a recording medium on which the computer program described in Supplementary Note 13 is recorded.

The present invention is not limited to the examples described above and is allowed to be changed, if desired, without departing from the essence or spirit of the invention which can be read from the claims and the entire specification. A patient condition prediction apparatus, a patient condition prediction method, and a computer program with such modifications are also intended to be within the technical scope of the present invention.

### Description of Reference Codes

1 Patient condition prediction apparatus
11 CPU
12 RAM
13 ROM
14 Memory apparatus
15 Input apparatus
16 Power apparatus
17 Data bus
110 Patient data acquisition unit
120 Predictive model selection unit
130 Patient condition prediction unit

What is claimed is:

1. A patient condition prediction apparatus comprising:
at least one memory that is configured to store informations; and
at least one processor that is configured to execute instructions
to obtain patient data, which are information about a patient;
to select one predictive model from a plurality of predictive models for predicting a change in a patient condition that is a condition of the patient, on the basis of the patient data; and
to predict a change in the patient condition in the future by using the one predictive model.

2. The patient condition prediction apparatus according to claim 1, wherein the processor predicts a risk of complications indicating a possibility of the patient developing complications, on the basis of the predicted change in the patient condition in the future.

3. The patient condition prediction apparatus according to claim 2, wherein the processor outputs information about treatment for the patient on the basis of the predicted risk of complications.

4. The patient condition prediction apparatus according to claim 1, wherein each of the plurality of predictive models is a model that allows prediction of the change in the patient condition in the future by using the patient condition in a first period that is defined for each of the predictive models and that is in the past.

5. The patient condition prediction apparatus according to claim 4, wherein the processor selects a model in which the first period is longer as a hospitalization period of the patient is longer.

6. The patient condition prediction apparatus according to claim 4, wherein the processor selects a model in which the first period is longer as the patient is younger in age.

7. The patient condition prediction apparatus according to claim 1, wherein each of the plurality of predictive models is a model that allows prediction of the change in the patient condition in a second period that is defined for each of the predictive models and that is in the future.

8. The patient condition prediction apparatus according to claim 7, wherein the processor selects a model in which the second period is longer as the patient condition needs to be grasped for a longer period.

9. The patient condition prediction apparatus according to claim 1 wherein the patient data includes an index that is defined by a degree of activities that can be performed by the patient.

10. The patient condition prediction apparatus according to claim 1 wherein the patient data includes information about a hospitalization period of the patient.

11. The patient condition prediction apparatus according to claim 1 wherein the patient data includes information about vital signs of the patient.

12. A patient condition prediction method comprising:
obtaining patient data, which are information about a patient;
selecting one predictive model from a plurality of predictive models for predicting a change in a patient condition that is a condition of the patient, on the basis of the patient data; and
predicting a change in the patient condition in the future by using the one predictive model.

13. A non-transitory recording medium on which a computer program that allows a computer to execute a patient condition prediction method is recorded, the patient condition prediction method including:
obtaining patient data, which are information about a patient;
selecting one predictive model from a plurality of predictive models for predicting a change in a patient condition that is a condition of the patient, on the basis of the patient data; and
predicting a change in the patient condition in the future by using the one predictive model.
